(19) United States
(12) Patent Application Publication
(10) Pub. No.: US 2019/0076628 A1
(43) Pub. Date: Mar. 14, 2019

(54) SPLITTABLE CATHETER INSERTION DEVICE AND RELATED METHODS

(71) Applicant: Osprey Vascular, LLC, Salt Lake City, UT (US)

(72) Inventor: Michael Anstett, New Port Richey, FL (US)

(21) Appl. No.: 15/981,769

(22) Filed: May 16, 2018

Related U.S. Application Data
(60) Provisional application No. 62/556,864, filed on Sep. 11, 2017.

(51) Int. Cl. A61M 25/06

(52) U.S. Cl. CPC A61M 25/0668; A61B 17/3415; A61M 25/065

(57) ABSTRACT

A splittable catheter insertion device includes a splittable housing that includes a body, a splitting seam extending along a longitudinal length of the body, a needle receiving, a pair of splitting grips extending outward from opposite lateral sides of the body, a channel formed within the body and extending along a portion of the longitudinal length of the body. The splittable catheter insertion device further includes a splittable needle secured to the needle receiving portion of the body, a pair of rails disposed within the channel and on opposite sides of the channel and extending in a direction parallel to the longitudinal length of the body, an advancer portion disposed within the channel, between the pair of rails, and configured to slide along the pair of rails, and a catheter predisposed within the splittable housing, attached to the advancer portion, and sized and shaped to slide through the splittable needle. Related methods are also disclosed.

### CROSS-REFERENCE TO RELATED APPLICATION

This application claims the benefit under 35 U.S.C. § 119(e) of U.S. Provisional Patent Application Ser. No. 62/556,864, filed Sep. 11, 2017, the disclosure of which is hereby incorporated herein in its entirety by this reference.

### FIELD

Embodiments of the present disclosure relate generally to catheter insertion devices, through-the-needle catheters, and related methods.

### BACKGROUND

Catheters are used in various medical procedures to administer fluids to a patient and/or to withdraw body fluids from a vein of a patient. Catheters are generally made of a flexible plastic material or various polymers, and a needle is used to access a vein or artery in the body in order to introduce the catheter into a blood vessel. One catheter insertion technique is known as the “through-the-needle” technique. In this technique, the needle is inserted through the skin and into the blood vessel until the needle tip is properly located in the vessel. Proper location of the needle tip is usually noted by a small flow of blood through the needle and into a flash chamber in a needle hub. After the needle is in proper position, a catheter is advanced through the needle and into the vessel. The needle is then withdrawn from the body, leaving the catheter implanted with the distal end of the catheter located in the vessel. However, typically, the needle cannot be easily removed and disposed of because of the interference of the catheter hub at the proximal end of the catheter. Accordingly, a common solution to this problem with the through-the-needle catheter is to remove the needle from the catheter by splitting the needle. For example, the needle may be made splittable by forming the needle of two longitudinally aligned halves, or by longitudinally scoring or perforating the body of the needle.

However, even with a splittable needle, portions of the catheter are often put in contact with the skin of patient prior to inserting the catheter into the patient's vein. Furthermore, the catheter is typically manipulated by an operator while threading the catheter through the lumen of the needle. As a result, catheters are often contaminated by bacteria from the skin of the patient or due to manipulation by the operator.

### BRIEF SUMMARY

Some embodiments of the present disclosure include splittable catheter insertion devices. The splittable catheter insertion devices may include a splittable housing, and the splittable housing may include a body, at least one preformed splitting seam extending along at least substantially an entire longitudinal length of the body, a needle receiving portion formed on a first longitudinal end of the body, a pair of splitting grips extending outward from opposite lateral sides of the body, a channel formed within the body and extending along a portion of the longitudinal length of the body and extending longitudinally from a second longitudinal end of the body. The splittable catheter insertion devices may further include a splittable needle secured to the needle receiving portion of the body, a pair of rails disposed within the channel and on opposite sides of the channel and extending in a direction parallel to the longitudinal length of the body, an advancer portion disposed within the channel, between the pair of rails, and configured to slide along the pair of rails, and a catheter predisposed within the splittable housing, attached to the advancer portion, and sized and shaped to slide through the splittable needle.

Further embodiments of the present disclosure include splittable catheter insertion devices according to other embodiments of the present disclosure. The splittable catheter insertion devices may include a splittable housing having a channel formed therein, the channel extending along a portion of a longitudinal length of the splittable housing, a splittable needle secured to a longitudinal end of the splittable housing, a pair of rails disposed within the channel, an advancer portion disposed within the channel and configured to slide along the pair of rails; and a catheter predisposed within the splittable housing, fitted to the advancer portion, and sized and shaped to slide through the splittable needle.

Additional embodiments of the present disclosure include methods of inserting catheters. The methods may include employing a splittable catheter insertion device including: a splittable housing having a channel formed therein, the channel extending along a portion of a longitudinal length of the splittable housing, a pair of splitting grips extending outward from opposite lateral sides of the splittable housing, a splittable needle secured to a longitudinal end of the splittable housing, a pair of rails disposed within the channel, an advancer portion disposed within the channel and configured to slide along the pair of rails, and a catheter predisposed within the splittable housing, fitted to the advancer portion, and sized and shaped to slide through the splittable needle. The methods may further include inserting the splittable needle into a patient, without contacting the catheter, advancing the catheter through the splittable needle and into the patient by advancing the advancer portion along the pair of rails, removing the splittable needle from the patient without contacting the catheter, while removing the splittable needle, splitting the splittable housing and the splittable needle utilizing the pair of splitting grips, and leaving a proximal portion of the catheter outside of the patient.

### BRIEF DESCRIPTION OF THE DRAWINGS

FIG. 1 is a perspective view of a splittable catheter insertion device according to one or more embodiments of the present disclosure;

FIG. 2A is a side view of the splittable catheter insertion device of FIG. 1;

FIG. 2B is a rear view of the splittable catheter insertion device of FIG. 1;

FIG. 2C is a top view of the splittable catheter insertion device of FIG. 1;

FIG. 2D is a side cross-sectional view of the splittable catheter insertion device of FIG. 1;

FIG. 3 is a perspective view of a rail system of the splittable catheter insertion device according to one or more embodiments of the present disclosure;

FIG. 4 is a side cross-sectional view of the splittable catheter insertion device having a rail system disposed therein according to one or more embodiments of the present disclosure; and

FIG. 5 is a perspective view of an advancer portion of a splittable catheter insertion device according to one or more embodiments of the present disclosure.

### DETAILED DESCRIPTION

The illustrations presented herein are not actual views of any particular catheter insertion device, but are merely idealized representations employed to describe example embodiments of the present disclosure. The following description provides specific details of embodiments of the present disclosure in order to provide a thorough description thereof. However, a person of ordinary skill in the art will understand that the embodiments of the disclosure may be practiced without employing many such specific details. Indeed, the embodiments of the disclosure may be practiced in conjunction with conventional techniques employed in the industry. In addition, the description provided below does not include all elements to form a complete structure or assembly. Only those process acts and structures necessary to understand the embodiments of the disclosure are described in detail below. Additional conventional acts and structures may be used. Also note, any drawings accompanying the application are for illustrative purposes only, and are thus not drawn to scale. Additionally, elements common between figures may have corresponding numerical designations.

As used herein, the terms “comprising,” “including,” and grammatical equivalents thereof are inclusive or open-ended terms that do not exclude additional, un-recited elements or method steps, but also include the more restrictive terms “consisting of,” “consisting essentially of,” and grammatical equivalents thereof.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, the term “configured” refers to a size, shape, material composition, and arrangement of one or more of at least one structure and at least one apparatus facilitating operation of one or more of the structure and the apparatus in a predetermined way.

As used herein, the singular forms following “a,” “an,” and “the” are intended to include the plural forms as well, unless the context clearly indicates otherwise.

As used herein, the term “and/or” includes any and all combinations of one or more of the associated listed items.

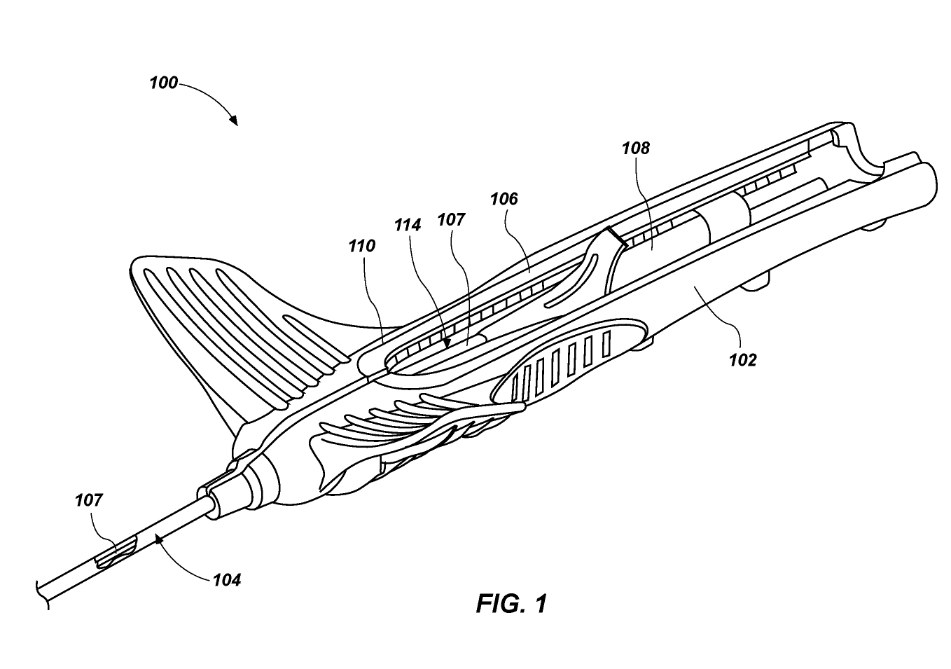

As used herein, spatially relative terms, such as “below,” “lower,” “bottom,” “above,” “upper,” “top,” and the like, may be used for ease of description to describe one element's or feature's relationship to another element(s) or feature(s) as illustrated in the figures. Unless otherwise specified, the spatially relative terms are intended to encompass different orientations of the materials in addition to the orientation depicted in the figures. For example, the spatially relative terms may refer to a catheter insertion device when the device is disposed on a horizontal surface (e.g., the position depicted in FIG. 1)

As used herein, the term “substantially” in reference to a given parameter, property, or condition means and includes to a degree that one of ordinary skill in the art would understand that the given parameter, property, or condition is met with a degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “vascular catheter” means and includes any catheter that may be used for providing access to the vasculature, such as one or more veins or one or more arteries of a patient, such as a midline catheter, a basilic catheter, a cephalic catheter, a centesis catheter (for deployment in the thoracic or abdominal regions of a patient), or another type of catheter. Vascular catheters described herein may comprise an arterial catheter or a venous catheter.

Embodiments of the present disclosure include a splittable vascular catheter insertion device that provides a “no touch” method of placing a catheter in a vein of a patient. In some embodiments, the splittable vascular catheter is placed at the basilic, cephalic, or median vein in the antecubital fossa. Additional embodiments include splittable vascular catheter insertion devices that provide a “no touch” method of placing an arterial catheter in an artery of a patient. Further embodiments include splittable vascular catheter insertion devices that provide a “no touch” method of placing a centesis catheter in an abdominal space or a thoracic space of a patient in order to enable draining of excess fluids from the patient.

For example, the splittable catheter insertion device (midline, arterial, venous, or centesis catheter insertion device, etc.) of the present disclosure enables an operator to place a catheter within a patient in a “no touch” method as referenced above, such method being implemented by the operator without physically touching the catheter and without contacting any portion of the catheter to the patient's skin prior to or while placing the catheter. For example, the splittable catheter insertion device of the present disclosure includes a splittable housing, a splittable needle, a catheter predisposed within the splittable housing (within a sterile environment), and an advancer portion. During operation, an operator may insert the splittable needle into a patient, advance the catheter with the advancer portion through the splittable needle, and remove the splittable needle while splitting the splittable housing and splittable needle. As a result, a proximal end of the catheter (e.g., a catheter hub) may be left exposed outside of the patient, and the catheter may be placed without the operator or the patient's skin ever directly contacting the catheter.

In view of the foregoing, the splittable catheter insertion device of the present disclosure may be advantageous over conventional catheter insertion devices. In particular, the catheter insertion device can introduce a catheter into the patient without the catheter ever touching (e.g., contacting, brushing, dragging across) the skin of the patient. Furthermore, the catheter can be introduced into the patient without an operator (e.g., a clinician) ever physically touching the catheter. As a result, the splittable catheter insertion device of the present disclosure can reduce the risk of contaminating the catheter prior to and while introducing the catheter to the patient. Moreover, the splittable catheter insertion device of the present disclosure can reduce the risk of infection, and as a result, can decrease sickness and disease that may be caused by contamination and infection.

Furthermore, because the catheter is a through-the-needle catheter, as will be understood by one of ordinary skill in the art, an operator does not need to change the angle of the needle after insertion in order to thread the catheter through the needle. This results in less pain to the patient, a faster catheter placement time, and a reduced risk of damaging the vessel accessed by the needle during insertion of the catheter. Additionally, the splittable catheter insertion device of the present disclosure may simplify a placement procedure of the catheter, may reduce clinical error (e.g., errors that would result in multiple attempts, increased risks of infection), and may reduce a risk of damaging the catheter (e.g., damage that can cause thrombogenesis and/or embolism of a piece of the catheter). Moreover, the splittable catheter insertion device removes any need for a guide wire for introducing the catheter.

FIG. 1 shows a perspective view of a splittable catheter insertion device 100 (hereinafter catheter insertion device 100) according to one or more embodiments of the present disclosure. In some embodiments, the catheter insertion device 100 may include a splittable housing 102, a splittable needle 104, a rail system 106, a predisposed catheter 107, and an advancer portion 108. As used herein, the term “splittable,” when used in reference to a member, indicates that portions of the member can be separated from each other. In some embodiments, the portions may comprise longitudinally aligned portions (e.g., two longitudinally aligned portions (halves)) of the member that can be separated from each other along a longitudinal length of the member. The splittable needle 104 may be secured (e.g., coupled) to a longitudinal end of the splittable housing 102. In some instances, at least a portion of the splittable needle 104 may be embedded within the longitudinal end of the splittable housing 102. In one or more embodiments, the splittable needle 104 may include any splittable needle 104 known in the art. For example, the splittable needle 104 may include a 17-gauge needle. In alternative embodiments, the splittable needle 104 may include any gauge of needle appropriate for the intended application of the through-needle catheter.

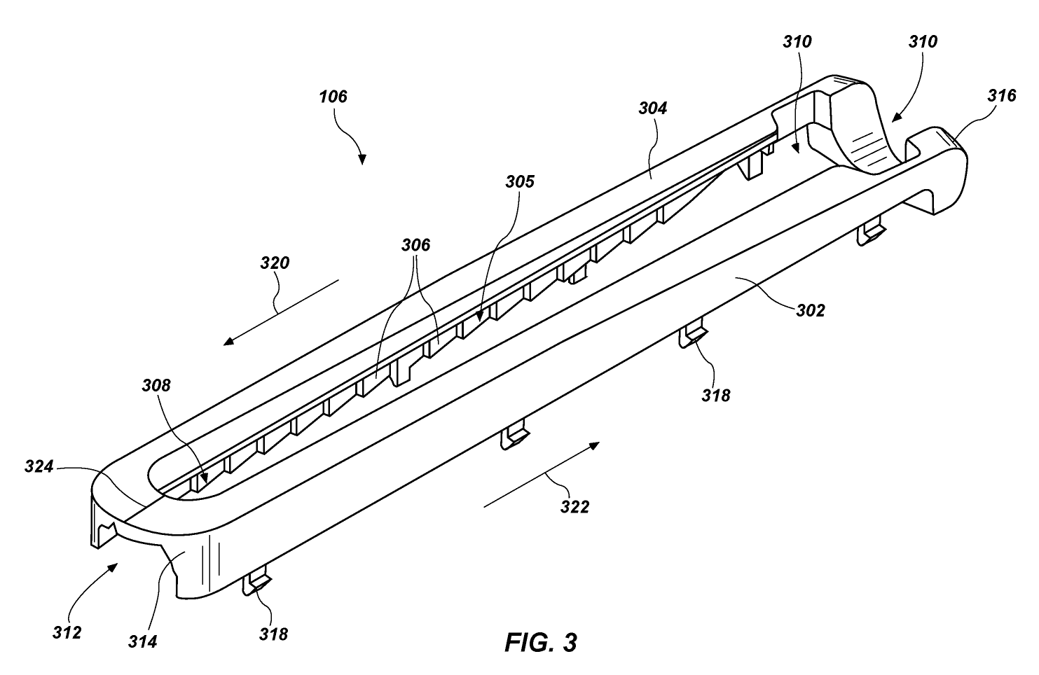

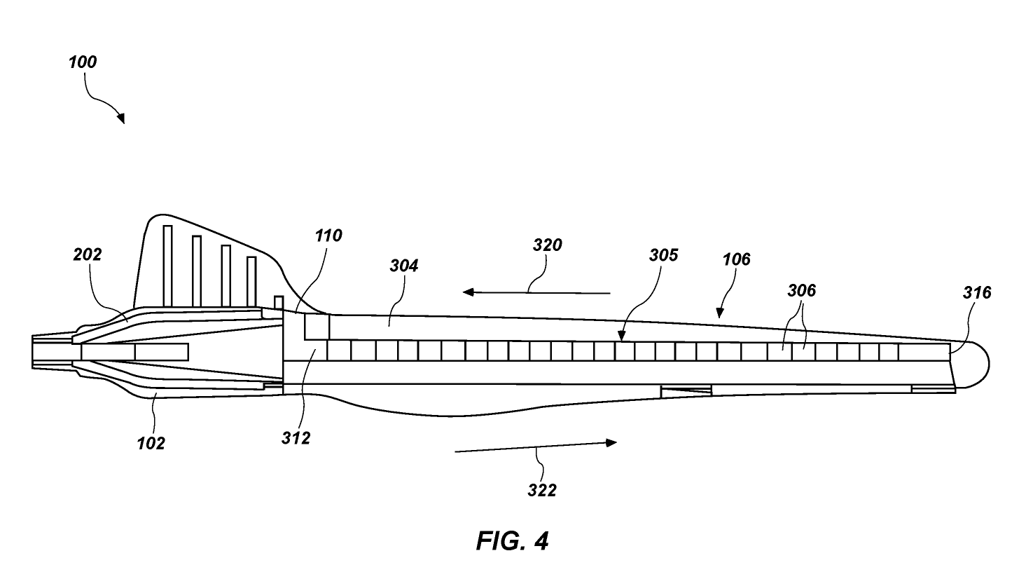

The splittable housing 102 may define a channel 110 extending along a longitudinal length of the splittable housing 102, and the rail system 106 may be at least partially disposed within the channel 110. For example, as is discussed in greater detail below in regard to FIGS. 3 and 4, the rail system 106 may be seated within the channel 110 and may be secured to the splittable housing 102. Additionally, the advancer portion 108 may be at least partially disposed within the rail system 106 and may be sized and shaped to slide along the rail system 106 during use.

The catheter 107 may be predisposed within catheter insertion device 100. For example, the catheter 107 may be predisposed within the splittable needle 104 and splittable housing 102 of the catheter insertion device 100. Furthermore, the catheter 107 may be coupled to the advancer portion 108. As used herein, the term “predisposed” when used in reference to a catheter 107 indicates that the catheter 107 was inserted into the catheter insertion device 100 under sterile conditions and prior to packaging of the sterile catheter 107 assembled with the catheter insertion device 100. Furthermore, the term “predisposed” may indicate that the catheter 107 was oriented in a “ready to use” position within the catheter insertion device 100, meaning that no manipulation of the catheter 107 is required by an operator (e.g., a clinician) prior to inserting the catheter 107 into a patient. In additional embodiments, the term “predisposed” may include that the catheter 107 (e.g., a non-sterile catheter) was inserted into the catheter insertion device 100 and was then sterilized prior to use.

In one or more embodiments, the advancer portion 108 may be part of the catheter 107. For example, the advancer portion 108 may form a connection member of the catheter 107 (i.e., a member for connecting the catheter 107 to other equipment). In additional embodiments, the advancer portion 108 may include a catheter hub of the catheter 107. In other embodiments, the advancer portion 108 may be separate and distinct from the catheter 107. Regardless, the catheter 107 may be formed from a flexible material. For example, the catheter 107 may be formed from one or more of silicone, vinyl, polyurethane, polyethylene, a TYGON® polymer, and polyvinyl chloride. Furthermore, the catheter 107 may have a gauge within the range of about 18 and about 22. For example, the catheter 107 may include a 20-gauge catheter. Additionally, the catheter 107 may have a length within a range of about 8 cm to about 20 cm. For instance, the catheter 107 may have a length of about 10 cm. Regardless, the catheter 107 may be sized and shaped to slide through a lumen of the splittable needle 104. In some embodiments, the catheter 107 may include a reverse taper or a bump (e.g., a portion of the catheter having an increased diameter). The reverse taper or bump may at least partially reduce (e.g., prevent or limit) leakage of blood (i.e., the patient's blood) through a space between an inner surface of the splittable needle 104 and an outer surface of the catheter 107. In other words, the reverse taper or bump may at least partially fill the space between the inner surface of the splittable needle 104 and the outer surface of the catheter 107. Furthermore, the reverse taper or bump provides additional structural support to the catheter 107. In some embodiments, the reverse taper or bump may be located at a proximal end (i.e., an end located away from the splittable needle 104) of the catheter 107.

In some embodiments, the catheter insertion device 100 may further include a flash window 114 for visual confirmation of proper needle access. For example, the flash window 114 may include a visible portion of the catheter 107 within the rail system 106 of the catheter insertion device 100. Furthermore, as will be understood by one of ordinary skill in the art, the catheter 107 may be extruded (e.g., formed) with a translucent or transparent material to allow visualization of blood within the catheter 107 (i.e., flash).

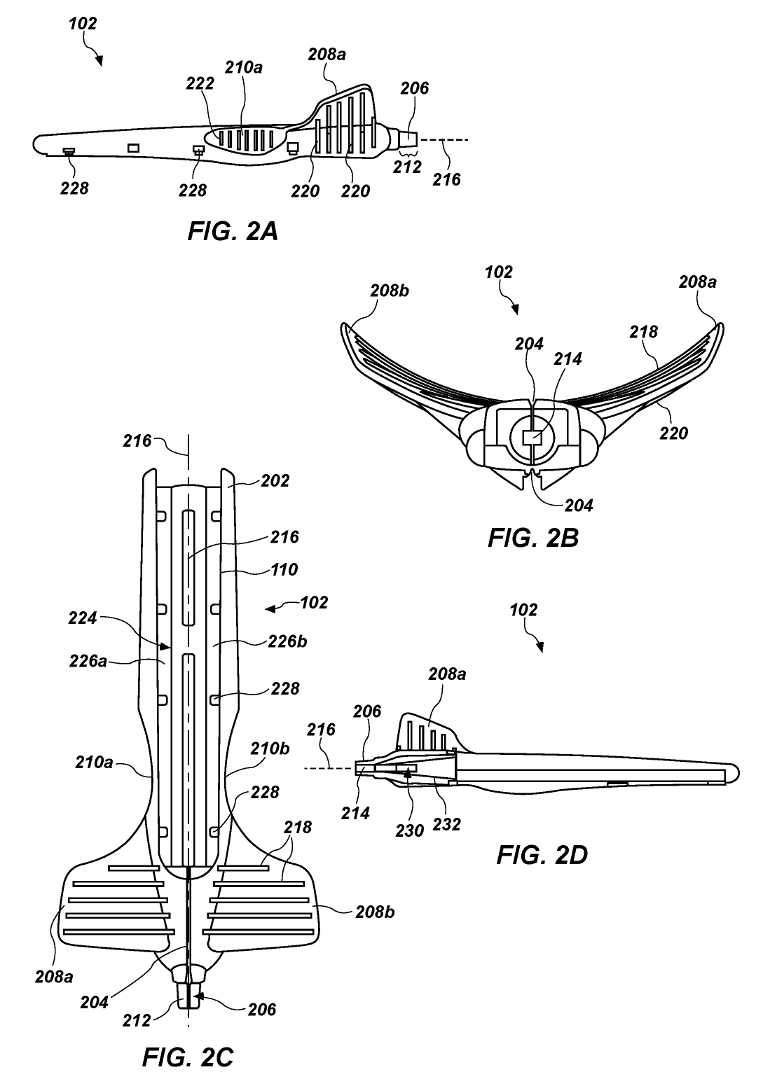

FIG. 2A is a side view of a splittable housing 102 of the catheter insertion device 100 according to one or more embodiments of the present disclosure. FIG. 2B is a back view of the splittable housing 102 of FIG. 2A. FIG. 2C is a top view of the splittable housing 102 of FIG. 2A. FIG. 2D is a side cross-sectional view of the splittable housing 102 of FIG. 2A. Referring to FIGS. 2A-2D together, the rail system 106 (FIG. 1) and advancer portion 108 (FIG. 1) are removed to better show the structure and elements of the splittable housing 102.

In some embodiments, the splittable housing 102 may include a body 202, a splitting seam 204, a needle-securing portion 206, a pair of splitting grips 208a, 208b, a pair of insertion grips 210a, 210b, and the channel 110. In some embodiments, the splitting seam 204 may include a narrowed portion of the splittable housing 102. In particular, the splitting seam 204 may include a thinner portion of an exterior wall of the splittable housing 102. In other embodiments, the splitting seam 204 may include scoring through the exterior wall of the body 202 of the splittable housing 102 and/or perforations extending through the exterior of the body 202 of the splittable housing 102. In some embodiments, the splitting seam 204 may extend linearly along a longitudinal length of the body 202 of the splittable housing 102. Furthermore, the splitting seam 204 may be at least substantially centered between lateral sides of the body 202 of the splittable housing 102. In some instances, the splitting seam 204 may extend along an upper wall of the body 202 and along a lower wall of the body 202 (e.g., along the exterior wall of the body 202). Furthermore, the splitting seam 204 may be sized and shaped to break (e.g., split) relatively easily. For example, as will be discussed in greater detail below, the splittable housing 102 may be splittable along the splitting seam 204.

The needle-securing portion 206 may be formed on a longitudinal end of the body 202 of the splittable housing 102. For purposes of the present disclosure, the needle-securing portion 206 will be referred to herein as being formed on a distal end of the body 202 of the splittable housing 102. Furthermore, a distal end of the body 202 may be an end of the body 202 that is proximate to a patient when the catheter insertion device 100 is utilized to place a catheter 107 in the patient. In some embodiments, the needle-securing portion 206 may be sized and shaped to have the splittable needle 104 (e.g., splittable needle 104 (FIG. 1)) extending therethrough. For example, in some instances, the needle-securing portion 206 may include a cylindrical portion 212 extending from longitudinal end of the body 202 of the splittable housing 102 and an aperture 214 extending through the cylindrical portion 212 along a center longitudinal axis 216 of the body 202. The aperture 214 may be sized and shaped to have the splittable needle 104 extend therethrough. In some embodiments, the splittable needle 104 may be molded with the splittable housing 102. For example, the splittable needle 104 may include an anchor portion (not shown) that is anchored (i.e., embedded) within the needle-securing portion 206 of the body 202 of the splittable housing 102.

The pair of splitting grips 208a, 208b may extend generally radially outward from the body 202 of the splittable housing 102. In some embodiments, each splitting grip of the pair of splitting grips 208a, 208b may have a generally relatively thin curved shape. For example, each splitting grip of the pair of splitting grips 208a, 208b may be secured to the body 202 of the splittable housing 102 and may generally curve upward relative to the body 202 of the splittable housing 102 as the splitting grip 208a, 208b extends away from the body 202 of the splittable housing 102. Furthermore, each splitting grip of the pair of splitting grips 208a, 208b may include one or more raised ridges extending along a length of the splitting grip (e.g., extending in a direction at least generally perpendicular to a center longitudinal axis 216 of the body 202). For instance, each splitting grip of the pair of splitting grips 208a, 208b may include a plurality of upper raised ridges 218 extending along an upper surface of the splitting grip and a plurality of lower raised ridges 220 extending along a lower surface of the splitting grip. In one or more embodiments, the pair of splitting grips 208a, 208b may be formed proximate to the needle-securing portion 206 of the splittable housing 102. In alternative embodiments, the pair of splitting grips 208a, 208b may be formed anywhere along a longitudinal length of the body 202 of the splittable housing 102. As is discussed in greater detail below, a user may utilize the pair of splitting grips 208a, 208b to grip the catheter insertion device 100 and split the catheter insertion device 100 along the splitting seam 204 after placing the predisposed catheter 107 into a patient.

Furthermore, the pair of insertion grips 210a, 210b may be formed on opposite lateral sides of the body 202 of the splittable housing 102. In some embodiments, each insertion grip of the pair of insertion grips 210a, 210b may include an indentation extending inward toward the center longitudinal axis 216 of the splittable housing 102. For example, each insertion grip of the pair of insertion grips 210a, 210b may include a general inverted semi-cylindrical shape. Furthermore, each insertion grip of the pair of insertion grips 210a, 210b may include one or more raised ridges 222 for gripping the splittable housing 102 when utilizing the catheter insertion device 100. In other embodiments, the pair of insertion grips 210a, 210b merely include the one or more raised ridges 222 on lateral sides of the body 202 and may not include indentations. In one or more embodiments, the pair of insertion grips 210a, 210b may be formed adjacent to the pair of splitting grips 208a, 208b. In other embodiments, the pair of insertion grips 210a, 210b may be formed anywhere along the longitudinal length of the splittable housing 102. As is discussed in greater detail below, a user may utilize the pair of insertion grips 210a, 210b to grip the catheter insertion device 100 when placing the splittable needle 104 into a patient, removing the splittable needle from the patient, and inserting the predisposed catheter 107 into the patient.

As mentioned briefly above, the splittable housing 102 may further define a channel 110 therein. In some embodiments, the channel 110 may extend downward into the body 202 of the splittable housing 102. Furthermore, the channel 110 may extend longitudinally completely through a longitudinal end (i.e., a proximal end) of the splittable housing 102 opposite the needle-securing portion 206. For example, the channel 110 may define a cutout in the longitudinal end (i.e., proximal end) of the body 202 of the splittable housing 102. In one or more embodiments, the channel 110 may define a seat 224 at a base of the channel 110 for receiving the rail system 106 (FIG. 1). The seat 224 may include a pair of ledges 226a, 226b extending inward from sidewalls of the channel 110 and a plurality of receiving apertures 228 extending through the pair of ledges 226a, 226b. In some embodiments, the splitting seam 204 may intersect the channel 110.

In some embodiments, the splittable housing 102 may further define a tapered aperture 230 having a tapered surface 232. The tapered aperture 230 may extend from the channel 110 of the splittable housing 102 to the needle-securing portion 206 of the splittable housing 102. The tapered surface 232 may have a smaller cross-sectional area proximate the distal end of the splittable housing 102 and may have a relatively increasing cross-sectional area as it extends toward the proximal end of the splittable housing 102. The tapered aperture 230 may serve to guide the catheter 107 (FIG. 1) as the catheter is inserted into a patient. Furthermore, the tapered aperture 230 may help prevent the catheter from kinking or bending while the catheter is placed in the patient.

FIG. 3 is a perspective view of the rail system 106 of a catheter insertion device 100 according to one or more embodiments of the present disclosure. FIG. 4 is a side cross-sectional view of a catheter insertion device 100 having the rail system 106 disposed therein. Referring to FIGS. 3 and 4 together, as shown, in some embodiments, the rail system 106 may include a single unitary piece that can be disposed within the channel 110 of the splittable housing 102. Furthermore, the rail system 106 may include an outer peripheral wall 302, an upper ledge 304, a pair of rails 305 (i.e., ratcheting rails) defining a plurality of teeth 306, a slide aperture 308, a receiving port 310, a catheter cutout notch 312, a first longitudinal end 314 (i.e., a distal end), a second opposite longitudinal end 316 (i.e., a proximal end), and a plurality of catch tabs 318. In one or more embodiments, the rail system 106 may have a generally elongated rounded rectangular shape (e.g., elongated pill shape).

The peripheral wall 302 may include a vertical wall and may extend around a periphery of the rail system 106. The upper ledge 304 may extend from a top of the peripheral wall 302 and may extend inward from the peripheral wall 302. The upper ledge 304 may define the slide aperture 308 between opposing portions of the upper ledge 304 (e.g., portions of the upper ledge 304 extending from opposing portions of the peripheral wall 302). The slide aperture 308 may extend along a longitudinal length of the rail system 106, and, as is discussed in greater detail below, may permit the advancer portion 108 (FIG. 1) to slide along the rail system 106.

In some embodiments, the receiving port 310 may extend through the upper ledge 304 proximate to the second opposite longitudinal end 316 of the rail system 106. Furthermore, the receiving port 310 may be sized and shaped to allow the advancer portion 108 (FIG. 1) to be inserted into (i.e., disposed within) the rail system 106. In some embodiments, the receiving port 310 may include two cutouts from opposing portions of the upper ledge 304 proximate the second longitudinal end 316 of the rail system 106 and a cutout extending through an upper portion of the peripheral wall 302 defining the second longitudinal end 316 of the rail system 106. In some embodiments, the cutout may be sized and shaped to receive one or both of a catheter hub during assembly of the splittable catheter insertion device 100, and the advancer portion 108.

The pair of rails 305 may be defined beneath and adjacent to the upper ledge 304 of the rail system 106 and may extend in a direction parallel to a longitudinal length of the body 202 of the splittable housing 102. Each rail of the pair of rails 305 may include multiple consecutively-oriented teeth 306 of the plurality of teeth 306. Furthermore, each tooth of the plurality of teeth 306 may extend inward from an inner surface of the peripheral wall 302. In some embodiments, the plurality of teeth 306 may extend from portions of the peripheral wall 302 defining lateral sides of the rail system 106. In some instances, the plurality of teeth 306 may include a plurality of notches. Moreover, the plurality of teeth 306 may be sized and shaped to catch on the advancer portion 108 (FIG. 1) in order to control how the advancer portion 108 slides along the rail system 106. For example, each tooth of the plurality of teeth 306 may have a general right triangle cross-section shape when viewed from a plane orthogonal to an outer vertical surface of the peripheral wall 302. In some embodiments, the plurality of teeth 306 of each rail of the pair of rails 305 may be oriented to form a plurality of consecutive inclined ramps along the inner surface of the peripheral wall 302. Due to the shape of the plurality of teeth 306 of the pair of rails 305, the plurality of teeth 306 may enable the advancer portion 108 to pass over the plurality of teeth 306 (i.e., slide along the pair of rails 305) in a first direction 320 (e.g., from a proximal end to a distal end of the rail system 106) while at least substantially preventing the advancer portion 108 from passing over the teeth 306 in a second opposite direction 322.

In other embodiments, each rail of the plurality of rails 305 may not include a plurality of teeth 306 but rather, may include an at least substantially planar rail. In such embodiments, the pair of rails 305 may enable the advancer portion 108 to slide along the pair of rails 305 in both the first direction 320 and the second direction 322.

The catheter cutout notch 312 may extend through the peripheral wall 302 of the rail system 106 at the first longitudinal end 314 (e.g., distal end) of the rail system 106. Furthermore, the catheter cutout notch 312 may allow the predisposed catheter 107 (FIG. 1) to extend therethrough and into the splittable needle 104 and to be inserted into a patient utilizing the advancer portion 108 (FIG. 1).

Referring to FIGS. 2A-4 together, the plurality of catch tabs 318 of the rail system 106 may extend downward from a bottom of the peripheral wall 302 of the rail system 106. Furthermore, the plurality of catch tabs 318 may correlate to the plurality of receiving apertures 228 of the splittable housing 102 and may be sized and shaped to extend through the plurality of receiving apertures 228 when the rail system 106 is disposed within the channel 110 of the splittable housing 102. For example, the plurality of catch tabs 318 and the plurality of receiving apertures 228 may be oriented relative to each other in at least substantially a same pattern. In some embodiments, the plurality of catch tabs 318 may be sized and shaped to catch the ledges of the channel 110 when the rail system 106 is disposed within the channel 110 of the splittable housing 102 and to secure to the rail system 106 to the splittable housing 102.

In some embodiments, the rail system 106 may include another splitting seam 324 extending across longitudinal ends (e.g., the first and second longitudinal ends 314 and 316, respectively) of the rail system 106 (e.g., along portions of the peripheral wall 302 and upper ledge 304 forming the longitudinal ends of the rail system 106). Furthermore, when the rail system 106 is disposed within the body 202 of the splittable housing 102, the another splitting seam 324 may align with the splitting seam 204 of the body 202.

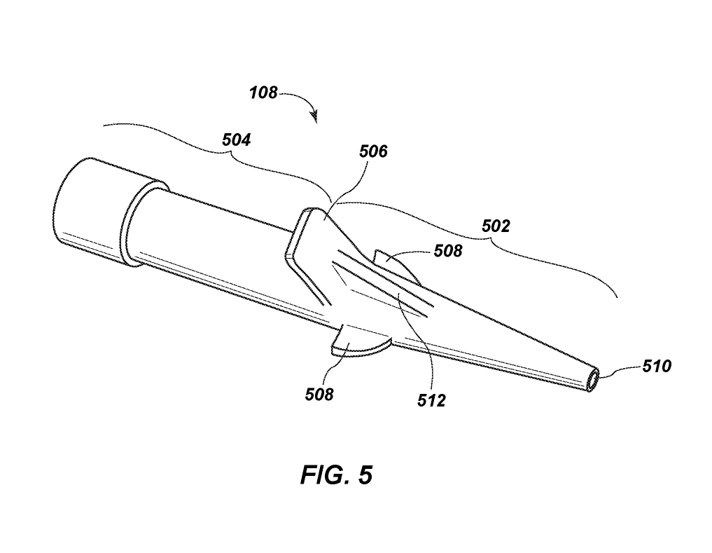

FIG. 5 is a perspective view of an advancer portion 108 according to one or more embodiments of the present disclosure. As noted above, in some instances, the advancer portion 108 may be a catheter hub of the predisposed catheter 107 (FIG. 1). In one or more embodiments, the advancer portion 108 may include a cylindrical portion 504, a frusto-conical portion 502 (i.e., a tapered portion), a push tab 506, a plurality of catch protrusions 508, and a central aperture 510.

The frusto-conical portion 502 may extend longitudinally from the cylindrical portion 504. In other words, the frusto-conical portion 502 and the cylindrical portion 504 may share a central longitudinal axis. Furthermore, the push tab 506 may extend generally radially outward from an intersection of the cylindrical portion 504 and the frusto-conical portion 502. In one or more embodiments, the push tab 506 may include a generally curved member curving toward the cylindrical portion 504 of the advancer portion 108. In some embodiments, the push tab 506 may include a support member 512 extending between a surface of the push tab 506 facing the frusto-conical portion 502 and the frusto-conical portion 502, such as from a surface of the push tab 506 along a portion of a surface of the frusto-conical portion 502. In some embodiments, the proximal end of the cylindrical portion 504 may be threaded, such as with female threads and may be configured to threadingly receive a component of a syringe or another component. In some such embodiments, the proximal end of the cylindrical portion 504 may comprise a luer lock configured to receive a luer lock syringe.

Although the advancer portion 108 is described herein as having a cylindrical portion 504 and a frusto-conical portion 502, the disclosure is not so limited. Rather, in one or more other embodiments, at least a portion of the cylindrical portion 504 may have a double-truncated circular cross-section. Furthermore, the advancer portion 108 may include any catheter hub known in the art with a push tab 506 attached thereto. In yet further embodiments, the advancer portion 108 may not include the frusto-conical portion 502. Rather, the advancer portion 108 may merely include a cylindrical portion 504. In some such embodiments, the push tab 506 may extend generally radially outward from a central portion of the cylindrical portion 504.

The plurality of catch protrusions 508 may also extend generally radially outward from a base portion of the frusto-conical portion 502 of the advancer portion 108. For example, a first catch protrusion 508 may extend radially outward from the frusto-conical portion 502 on a first lateral side of the advancer portion 108, and a second catch protrusion 508 may extend radially outward from the frusto-conical portion 502 on a second opposite lateral side of the advancer portion 108. In one or more embodiments, the plurality of catch protrusions 508 may extend in a direction that is at least substantially perpendicular to a direction in which the push tab 506 generally extends. In one or more embodiments, each catch protrusion 508 of the plurality of catch protrusions 508 may have a generally quarter-circle shape with a curved portion and surface of the catch protrusion 508 facing the frusto-conical portion 502 of the advancer portion 108 and a flat portion of the catch protrusion 508 facing the cylindrical portion 504 of the advancer portion 108. In some embodiments, the catch protrusions 508 may comprise a relatively flexible material, such as, for example, a silicone-based material.

A central aperture 510 may extend along a central longitudinal axis of the advancer portion 108 and may extend completely through the advancer portion 108. As noted above, in some embodiments, the advancer portion 108 may include a catheter hub, and, in such embodiments, the central aperture 510 of the advancer portion 108 may permit fluids being administered to a patient to flow therethrough and into the catheter 107.

Referring to FIGS. 1-5 together, in operation, in some embodiments, the catheter 107 (i.e., the predisposed catheter 107) may be predisposed within the splittable housing 102 and may be fitted to (e.g., fitted within) the frusto-conical portion 502 of the advancer portion 108. For example, the catheter 107 may be predisposed within the splittable housing 102 in a sterile environment and prior to packaging. In additional embodiments, as noted above, the catheter 107 may be predisposed within the splittable housing 102 in a non-sterile environment and then may be sterilized pre- or post-packaging. In embodiments where the advancer portion 108 includes a catheter hub of the catheter 107, the advancer portion 108 and the catheter 107 may be disposed together within the splittable housing 102. For example, the advancer portion 108 may be inserted through the receiving port 310 of the rail system 106. Moreover, the advancer portion 108 may be engaged with the rail system 106. For instance, the advancer portion 108 may be coupled to the pair of rails 305 of the rail system 106. In particular, the plurality of catch protrusions 508 of the advancer portion 108 may be engaged with the plurality of teeth 306 of the pair of rails 305. Furthermore, the rail system 106 may be disposed within the channel 110 of the splittable housing 102, and the another splitting seam 324 of the rail system 106 may be aligned with the splitting seam 204 of the splittable housing 102 of the catheter insertion device 100. As noted above, in some embodiments, the foregoing acts may be performed prior to packaging and in a sterile environment. Accordingly, a package or a kit may include the splittable catheter insertion device 100 with the catheter 107 predisposed within the splittable house 102, fitted to the frusto-conical portion 502 of the advancer portion 108, wherein rail system 106 is disposed within the channel 110 of the splittable housing 102 and the advancer portion 108 is inserted through the receiving port 310 of the rail system 106.

Upon opening the packaging, an operator may grasp the catheter insertion device 100 with the pair of insertion grips 210a, 210b and may insert the splittable needle 104 into a patient. After the splittable needle 104 is inserted into the patient and a location of the splittable needle 104 is confirmed (such as by visible confirmation of blood in the catheter 107) via the flash window 114, the catheter 107 (i.e., the predisposed catheter 107) may be advanced (e.g., pushed through the splittable needle 104 and into the patient) by utilizing the advancer portion 108. For example, as user may push against the push tab 506 of the advancer portion 108 to cause the advancer portion 108 to slide along the rail system 106 of the catheter insertion device 100. In some embodiments, the user may cause the advancer portion 108 to slide in a direction extending from the proximal end of the catheter insertion device 100 to a distal end of the catheter insertion device 100 (i.e., in a forward direction).

Causing the advancer portion 108 to slide along the rail system 106 may cause the catch protrusions 508 of the advancer portion 108 to slide along the plurality of teeth 306 of the pair of rails 305 of the rail system 106. Furthermore, as noted above, in some instances, the plurality of teeth 306 may at least substantially prevent the advancer portion 108 from sliding in a direction extending from the distal end of the catheter insertion device 100 to a proximal end of the catheter insertion device 100 (i.e., in a backward direction). In particular, for a given catch protrusion 508 of the plurality of catch protrusions 508, the curved portion (e.g., the curved surface) of the given catch protrusion 508 may slide along an inclined surface of a respective tooth 306, pass over a peak of the respective tooth 306, and slide into a position past a flat rear side of the respective tooth 306. In such a position, the flat portion of the given catch protrusion 508 may be adjacent to and/or may abut against the flat rear side of the respective tooth 306. Furthermore, because the flat portion of the given catch protrusion 508 abuts against the flat rear side of the respective tooth 306, the respective tooth 306 may at least substantially prevent the given catch protrusion 508 from passing over the respective tooth 306 in an opposite direction due to mechanical interference between the given catch protrusion 508 and respective tooth 306.

The advancer portion 108 may be advanced until the catheter 107 is placed within the patient. In some embodiments, after the catheter 107 (e.g., a distal end of the catheter 107) has been placed within the patient, the splittable needle 104 may be withdrawn from the patient. Furthermore, in one or more embodiments, while the splittable needle 104 is being withdrawn from the patient, a user may utilize the pair of splitting grips 208a, 208b to split the splittable housing 102 along the splitting seam 204. For example, a user can grasp the splitting grips 208a, 208b and can twist (e.g., pivot) the splitting grips 208a, 208b at least substantially about the center longitudinal axis 216 of the splittable housing 102 to cause the splittable housing 102 and rail system 106 to split along the splitting seam 204 of the splittable housing 102 and the another splitting seam 324 of the rail system 106. Additionally, the user may utilize the pair of splitting grips 208a, 208b to split the splittable needle 104 while withdrawing the splittable needle 104 from the patient. As a result, the catheter insertion device 100 can be split while a proximal end of the catheter 107 (e.g., the advancer portion 108 and a portion of the catheter 107) is left exposed outside of the patient.

In view of the foregoing, the catheter insertion device 100 of the present disclosure may be advantageous over conventional catheter insertion devices. In particular, the catheter insertion device 100 can introduce a catheter 107 into the patient without the catheter 107 ever touching (e.g., contacting, brushing, dragging across) the skin of the patient. Furthermore, the catheter 107 can be introduced into the patient without an operator (e.g., a clinician) physically touching the catheter 107. As a result, the catheter insertion device 100 of the present disclosure can reduce the risk of contaminating the catheter 107 prior to and while introducing the catheter 107 to the patient. Moreover, the catheter insertion device 100 of the present disclosure can reduce the risk of infection, and as a result, can decrease sickness that may be caused by contamination and infection.

Furthermore, because the catheter 107 is a through-the-needle catheter 107, as will be understood by one of ordinary skill in the art, an operator does not need to change the angle of the needle after insertion in order to thread the catheter 107 through the needle. This results in less trauma and pain to the patient and a faster catheter placement time. Additionally, as noted above, the splittable catheter insertion device of the present disclosure may simplify a placement procedure of the catheter, may reduce clinical error (e.g., errors that would result in multiple attempts, increased risks of infection), and may reduce a risk of damaging the catheter (e.g., damage that can cause thrombogenesis and/or embolism of a piece of the catheter).

In addition, the catheter insertion device 100 of the present disclosure is ready for use out of the package. For example, as discussed above, the catheter 107 is predisposed within the catheter insertion device 100, and upon opening the package the catheter insertion device 100 is ready for use. As a result, the catheter insertion device 100 of the present disclosure, in comparison to conventional catheter insertion devices, does not require any additional assembly and may require less time to place a catheter 107.

Referring to FIGS. 1-4 together, although the splittable housing 102 and the rail system 106 are described above as including two separate pieces, the disclosure is not so limited. For example, in some embodiments, the splittable housing 102 and the rail system 106 may include a single unitary body. For instance, the splittable housing 102 and rail system 106 may include a single piece.

Additional non-limiting example embodiments of the disclosure are described below.

### Embodiment 1

A splittable catheter insertion device, comprising: a splittable housing comprising: a body; at least one preformed splitting seam extending along at least substantially an entire longitudinal length of the body; a needle receiving portion formed on a first longitudinal end of the body; a pair of splitting grips extending outward from opposite lateral sides of the body; a channel formed within the body and extending along a portion of the longitudinal length of the body and extending longitudinally from a second longitudinal end of the body; a splittable needle secured to the needle receiving portion of the body; a pair of rails disposed within the channel and on opposite sides of the channel and extending in a direction parallel to the longitudinal length of the body; an advancer portion disposed within the channel, between the pair of rails, and configured to slide along the pair of rails; and a catheter predisposed within the splittable housing, attached to the advancer portion, and sized and shaped to slide through the splittable needle.

### Embodiment 2

The splittable catheter insertion device of Embodiment 1, wherein the at least one preformed splitting seam of the body intersects the channel of the body.

### Embodiment 3

The splittable catheter insertion device of Embodiments 1 or 2, wherein the splittable housing further comprises a pair of insertion grips formed on opposite lateral sides of the body.

### Embodiment 4

The splittable catheter insertion device of Embodiments 1, 2, or 3, wherein each rail of the pair of rails comprises a ratchet rail comprising a plurality of teeth.

### Embodiment 5

The splittable catheter insertion device of Embodiment 4, wherein the pair of rails comprises a single unitary piece disposable within the channel.

### Embodiment 6

The splittable catheter insertion device of Embodiment 5, wherein the single unitary piece defines a receiving port for receiving the advancer portion into the channel.

### Embodiment 7

The splittable catheter insertion device of Embodiments 4 or 5, wherein the advancer portion includes at least two catch protrusions extending radially outward from the advancer portion on opposite lateral sides of the advancer portion and sized and shaped to catch on teeth of the plurality of teeth of the pair of rails.

### Embodiment 8

The splittable catheter insertion device of Embodiments 1, 2, 3, 4, 5, 6, or 7, wherein the advancer portion comprises: a cylindrical portion; a frusto-conical portion extending from the cylindrical portion and sized and shaped to have the predisposed catheter fitted to a distal end of the frusto-conical portion; and a push tab extending radially outward from an intersection of the frusto-conical portion and the cylindrical portion and extending out of the channel of the splittable housing.

### Embodiment 9

The splittable catheter insertion device of Embodiments 1, 2, 3, 4, 5, 6, 7, or 8, wherein the advancer portion is configured to slide in only one direction along the pair of rails.

### Embodiment 10

A splittable catheter insertion device, comprising: a splittable housing having a channel formed therein, the channel extending along a portion of a longitudinal length of the splittable housing; a splittable needle secured to a longitudinal end of the splittable housing; a pair of rails disposed within the channel; an advancer portion disposed within the channel and configured to slide along the pair of rails; and a catheter predisposed within the splittable housing, fitted to the advancer portion, and sized and shaped to slide through the splittable needle.

### Embodiment 11

The splittable catheter insertion device of Embodiment 10, wherein each rail of the pair of rails is disposed along a respective lateral side of the channel and extends in a direction parallel to a central longitudinal axis of the splittable housing.

### Embodiment 12

The splittable catheter insertion device of Embodiments 10 or 11, wherein each rail of the pair of rails comprises a plurality of teeth, each tooth of the plurality of teeth having a right triangle cross-section.

### Embodiment 13

The splittable catheter insertion device of Embodiments 10, 11, or 12, wherein the splittable housing comprises a pair of splitting grips extending outward from opposite lateral sides of a body of the splittable housing and being disposed proximate to the splittable needle.

### Embodiment 14

The splittable catheter insertion device of Embodiments 10, 11, 12, or 13, wherein the splittable housing comprises a pairs of insertion grips formed on opposite lateral sides of a body of the splittable housing and comprising curved indentations.

### Embodiment 15

The splittable catheter insertion device of Embodiments 10, 11, 12, 13, or 14, wherein the pair of rails comprises a single unitary piece disposable within the channel.

### Embodiment 16

A method of inserting a catheter, the method comprising: employing a splittable catheter insertion device comprising: a splittable housing having a channel formed therein, the channel extending along a portion of a longitudinal length of the splittable housing; a pair of splitting grips extending outward from opposite lateral sides of the splittable housing; a splittable needle secured to a longitudinal end of the splittable housing; a pair of rails disposed within the channel; an advancer portion disposed within the channel and configured to slide along the pair of rails; and a catheter predisposed within the splittable housing, fitted to the advancer portion, and sized and shaped to slide through the splittable needle; inserting the splittable needle into a patient; without contacting the catheter, advancing the catheter through the splittable needle and into the patient by advancing the advancer portion along the pair of rails; removing the splittable needle from the patient without touching the catheter; while removing the splittable needle, splitting the splittable housing and the splittable needle utilizing the pair of splitting grips; and leaving a proximal end of the catheter outside of the patient.

### Embodiment 17

The method of Embodiment 16, wherein advancing the advancer portion along the pair of rails comprises: pushing on a push tab of the advancer portion; and causing the advancer portion to slide along the pair of rails.

### Embodiment 18

The method of Embodiments 16 or 17, wherein splitting the splittable housing and the splittable needle comprises splitting the splittable housing and the splittable needle utilizing the pair of splitting grips.

### Embodiment 19

The method of Embodiments 16, 17, or 18, wherein splitting the splittable housing and the splittable needle utilizing the pair of splitting grips comprises at least partially pivoting the splitting grips about a central longitudinal axis of the splittable housing.

### Embodiment 20

The method of Embodiments 16, 17, 18, or 19, wherein advancing the advancer portion along the pair of rails comprises causing at least one catch protrusion of the advancer portion to slide along a plurality of teeth of the pair of rails.

While the present invention has been described herein with respect to certain illustrated embodiments, those of ordinary skill in the art will recognize and appreciate that it is not so limited. Rather, many additions, deletions, and modifications to the illustrated embodiments may be made without departing from the scope of the invention as claimed, including legal equivalents thereof. In addition, features from one embodiment may be combined with features of another embodiment while still being encompassed within the scope of the invention as contemplated by the inventors. Further, embodiments of the disclosure have utility with different and various tool types and configurations.

What is claimed is:

1. A splittable catheter insertion device, comprising:
a splittable housing comprising:
a body;
at least one preformed splitting seam extending along at least substantially an entire longitudinal length of the body;
a needle receiving portion formed on a first longitudinal end of the body;
a pair of splitting grips extending outward from opposite lateral sides of the body;
a channel formed within the body and extending along a portion of the longitudinal length of the body and extending longitudinally from a second longitudinal end of the body;
a splittable needle secured to the needle receiving portion of the body;
a pair of rails disposed within the channel and on opposite sides of the channel and extending in a direction parallel to the longitudinal length of the body;
an advancer portion disposed within the channel, between the pair of rails, and configured to slide along the pair of rails; and
a catheter predisposed within the splittable housing, attached to the advancer portion, and sized and shaped to slide through the splittable needle.

2. The splittable catheter insertion device of claim 1, wherein the at least one preformed splitting seam of the body intersects the channel of the body.

3. The splittable catheter insertion device of claim 1, wherein the splittable housing further comprises a pair of insertion grips formed on opposite lateral sides of the body.

4. The splittable catheter insertion device of claim 1, wherein each rail of the pair of rails comprises a ratchet rail comprising a plurality of teeth.

5. The splittable catheter insertion device of claim 4, wherein the pair of rails comprises a single unitary piece disposable within the channel.

6. The splittable catheter insertion device of claim 5, wherein the single unitary piece defines a receiving port for receiving the advancer portion into the channel.

7. The splittable catheter insertion device of claim 4, wherein the advancer portion includes at least two catch protrusions extending radially outward from the advancer portion on opposite lateral sides of the advancer portion and sized and shaped to catch on teeth of the plurality of teeth of the pair of rails.

8. The splittable catheter insertion device of claim 1, wherein the advancer portion comprises:
a cylindrical portion;
a frusto-conical portion extending from the cylindrical portion and sized and shaped to have the catheter fitted to a distal end of the frusto-conical portion; and
a push tab extending radially outward from an intersection of the frusto-conical portion and the cylindrical portion and extending out of the channel of the splittable housing.

9. The splittable catheter insertion device of claim 1, wherein the advancer portion is configured to slide in only one direction along the pair of rails.

10. A splittable catheter insertion device, comprising:
a splittable housing having a channel formed therein, the channel extending along a portion of a longitudinal length of the splittable housing;
a splittable needle secured to a longitudinal end of the splittable housing;
a pair of rails disposed within the channel;
an advancer portion disposed within the channel and configured to slide along the pair of rails; and
a catheter predisposed within the splittable housing, fitted to the advancer portion, and sized and shaped to slide through the splittable needle.

11. The splittable catheter insertion device of claim 10, wherein each rail of the pair of rails is disposed along a respective lateral side of the channel and extends in a direction parallel to a central longitudinal axis of the splittable housing.

12. The splittable catheter insertion device of claim 10, wherein each rail of the pair of rails comprises a plurality of teeth, each tooth of the plurality of teeth having a right triangle cross-sectional shape.

13. The splittable catheter insertion device of claim 10, wherein the splittable housing comprises a pair of splitting grips extending outward from opposite lateral sides of a body of the splittable housing and being disposed proximate to the splittable needle.

14. The splittable catheter insertion device of claim 10, wherein the splittable housing comprises a pair of insertion grips formed on opposite lateral sides of a body of the splittable housing and comprising curved indentations.

15. The splittable catheter insertion device of claim 10, wherein the pair of rails comprises a single unitary piece disposable within the channel.

16. A method of inserting a catheter, the method comprising:
employing a splittable catheter insertion device comprising:
a splittable housing having a channel formed therein, the channel extending along a portion of a longitudinal length of the splittable housing;
a pair of splitting grips extending outward from opposite lateral sides of the splittable housing;
a splittable needle secured to a longitudinal end of the splittable housing;
a pair of rails disposed within the channel;
an advancer portion disposed within the channel and configured to slide along the pair of rails; and
a catheter predisposed within the splittable housing, fitted to the advancer portion, and sized and shaped to slide through the splittable needle;
inserting the splittable needle into a patient;
without contacting the catheter, advancing the catheter through the splittable needle and into the patient by advancing the advancer portion along the pair of rails;
removing the splittable needle from the patient without contacting the catheter;
while removing the splittable needle, splitting the splittable housing and the splittable needle utilizing the pair of splitting grips; and
leaving a proximal portion of the catheter outside of the patient.

17. The method of claim 16, wherein advancing the advancer portion along the pair of rails comprises:
pushing on a push tab of the advancer portion; and
causing the advancer portion to slide along the pair of rails.

18. The method of claim 16, wherein splitting the splittable housing and the splittable needle comprises splitting the splittable housing and the splittable needle utilizing the pair of splitting grips.

19. The method of claim 18, wherein splitting the splittable housing and the splittable needle utilizing the pair of splitting grips comprises at least partially pivoting each splitting grip of the pair of splitting grips about a central longitudinal axis of the splittable housing.

20. The method of claim 16, wherein advancing the advancer portion along the pair of rails comprises causing at least one catch protrusion of the advancer portion to slide along a plurality of teeth of the pair of rails.
